(19) United States
(12) Patent Application Publication
(10) Pub. No.: US 2020/0383693 A1
(43) Pub. Date: Dec. 10, 2020

(54) Parallel to Face Sculpted Edge Tongue Scrapper with Dental Flosser and Dental Pick

(71) Applicants: Hanna Jun, Snoqualmie, WA (US); Jae Jun, Snoqualmie, WA (US)

(72) Inventors: Hanna Jun, Snoqualmie, WA (US); Jae Jun, Snoqualmie, WA (US)

(73) Assignee: Open Arms Holdings LLC, Snoqualmie, WA (US)

(21) Appl. No.: 16/432,991

(22) Filed: Jun. 6, 2019

(51) Int. Cl. A61B 17/24; A61C 15/02; A61C 15/04

(52) U.S. Cl. CPC A61B 17/244; A61C 15/046; A61C 15/02

(57) ABSTRACT

A oral hygiene implement for cleaning the surface of a tongue, cleaning between teeth, and for massaging the gums at the tooth gum interface comprising a handle, a sculpted top edge with the sculpted potion covering the majority of the top edge, with a plastic dental flossing pick on one end of the device and a floss holding assembly having fork-shaped tines for holding floss therebetween on the other end of the device.

### RELATED PATENT APPLICATIONS

### US Patents

| Pat. No. | Kind Code | Issue Date | Patentee |
|---|---|---|---|
| 6,152,939 | A | Nov. 28, 2000 | Thuy B. Ngyen |
| 6,273,719 | B1 | Aug. 14, 2001 | Nathan Lane Whitman |
| 6,363,949 | B1 | Apr. 2, 2002 | Thomas W. Brown |
| 6,440,140 | B1 | Aug. 27, 2002 | Dasan Potti |

### US Patent Applications

| Publication # | Kind Code | Pub. Date | Applicant |
|---|---|---|---|
| 20040040572 | A1 | Mar. 4, 2004 | Ingram S, Chodrow |
| 20080185016 | A! | Aug. 7, 2008 | John M. Jansheski |
| | | | Lex Shankle |

### BACKGROUND—PRIOR ART

### 1. Field of the Invention

This invention is in the field of dental hygiene devices, including dental flossers, tongue scrapers and dental picks.

### 2. Background of the Invention Field

Most people brush their teeth perhaps once a day, floss between their teeth perhaps twice a week, and clean their tongue occasionally. Another dental hygiene tool used sporadically is a dental pick

Diligent people brush their teeth one or more times a day and floss at least once a day and frequently scrape their tongues. People dental care also use a dental plastic floss pick to clean between the teeth and also to gently press against the gum. Using the dental plastic floss picks with an in and out motion to remove food particles, plaque and stimulate gums. While dental flossers and tongue scrapers have been combined, applicant did not discover a similar three-in-one product in searches.

The invention of this application combines flossing, tongue scraping and the use of a plastic dental floss pick into one tool. The major important advancement is the use of a continuous sculpted edge on the device's handle, which creates a ruffled effect on the edge for tongue scraping. Those ruffles are what makes the device of this application effective to scrape the tongue to remove the white film from a dirty tongue. The straight edge of traditional tongue scrapers only glides over the surface of the tongue and does not dig in to remove the white film effectively. The ruffled edges act like a comb to dig into the surface and makes this device very effective.

### DESCRIPTION OF PRIOR ART

Prior art includes products with a combination of dental flossers and tongue scrapers, with at least one tongue scraper having an abrasive surface on the edge to improve efficacy, but it is not nearly the same as this device's continuous sculpted edge that creates the ruffle effect. The patents closest to the concept of this device are listed below.
1. U.S. Pat. No. 7,997,287, A dental flosser having a tongue cleaner, a rugous surface on at least one of the arms suitable for being scraped against the tongue of a user for effectively cleaning the tongue, This device does not have a pick, but its key feature is a rugous surface on the tongue scraper. A rugous surface is defined as a having a rough, ridged, or wrinkled surface. One claim cites as additional embodiment, having the rugous surface on raised ridges. This is not the same as this device which has a sculpted scraper which creates a furled effect which acts like a comb, ruffling the surface, and also the sculpted features of the device in this application are continuous. The sculpted surface of the tongue scraper is also much taller than a rugous surface and offers a much stronger comb like effect.
2. US 20040040572 offers a combination tongue scraper and dental flosser, with the dental flosser on one end and the tongue scraper on the other, and a handle in between. This device uses a standard smooth edge tongue scraper and does not have a dental pick.
3. U.S. Pat. No. 6,152,939 is a tongue scraper only, that functions parallel to the face, similar to the device of this invention, where either end is positioned near the corner of the mouth, the tongue is stuck out, and the tongue scraper pulled forward. The scraping portion of the product is a flexible strip, with abrasions on it, with the flexible strip having handles on either end.
4. U.S. Pat. No. 627,719 is a toothbrush and tongue scraper combination, with the two on either end of a handle. The tongue scraper is a continuous loop integrally formed with the handle at the second end of the handle and having an edge suitable for scraping the tongue. The tongue scraper in this patent has a traditional smooth edge available in most tongue scrapers today.
5. U.S. Pat. No. 6,363,949 is a combination tongue scraper and dental flosser. The tongue scraper here is a traditional loop with a smooth edge. The innovative feature is that the device containers a dental floss dispenser in addition to a dental flosser.
6. U.S. Pat. No. 6,440,149 is a dental flosser and tongue scrubber combination with the dental flosser and tongue scraper at opposite ends of the handle. In this case the innovative feature is the tongue scraper. The tongue scraper first has two smooth scrapers with one being on the outer edge of the device, and one slightly inward of the outer scraper. The disclosed device has two cutouts, but their purpose is uncertain, and the cutouts are not mentioned in the claims.

### SUMMARY OF THE INVENTION

A tongue and tooth cleaning device for cleaning the surface of a tongue, cleaning between teeth, and for massaging the gums at the tooth gum interface comprising a handle, a continuous sculpted top edge with the sculpted portion covering the majority of the top edge, with a plastic dental flossing pick on one end of the device and a floss holding assembly having fork-shaped fines for holding floss therebetween on the other end of the device. Additional embodiments include a shorter sculpted edge, varying curvatures in the sculpted edge tongue scraper, and variations in the shape of the handle and the width of the floss fork.

### OPERATION OF THE INVENTION

The dental flosser operates conventionally. The tongue scraper works primarily by the person sticking their tongue out, the tongue scraper going from one corner of the mouth toward the other end of the mouth and then being drawn the end of the tongue protruding from the mouth. The plastic dental flosser pick can be used to clean debris from areas between teeth, and also may be rubbed in the area between gum line and the teeth.

### ADVANTAGES OF THE INVENTION

The most prominent advantages of the invention are:
1. Three dental cleaning features combined in one device: a dental flosser, tongue scraper and plastic floss pick.
2. An improved surface for tongue cleaning, a continuous sculpted edge that creates a ruffled edge, providing a comb-like effect for great efficacy.

### COMPARISON TO RELATED INVENTION

This application is filed on the same day as the invention of the device Forward Pulling Sculpted Edge Tongue Scraper with Dental Flosser and Dental Floss Pick. Both devices rely on the same continuous sculpted edge of the tongue scraper which creates a ruffled effect to have a comb like effect on the tongue. The difference between the two are a different location for the sculpted edge, this device is on the top edge of the device, the Forward Pulling Sculpted Tongue Scraper on one end of the handle. That device also has a cutout to help remove debris, and the dental floss pick rests against the handle until it is rotated into position by the user who needs to twist the handle to create this effect.

### DRAWINGS—FIGURES

FIG. 1 is a ¾ view of the device, showing the sculpted edge along with the other features of the device.

FIG. 2 is a view of the device from the bottom, with the floss on dental flosser facing down.

FIG. 3 is a ¾ view of the device, showing more of a side view the of the sculpted edge.

FIG. 4 is a top view of the device showing the sculpted edge.

FIG. 5 is a front view of the device showing that the sculpted edge rises away from the dental flosser

FIG. 6 is a back view of the device showing that pick is only slightly below the arc of the sculpted edge.

### DRAWINGS—REFERENCE NUMBERS

1. Dental floss pick.

2. Sculpted edge tongue scraper.

3. The tine of the floss fork with a floss holding mechanism that is not on the end of the device.

4. The tine of the floss fork with a floss holding mechanism that is near the end of the device.

5. Dental floss

6. Handle.

### DETAILED DESCRIPTION OF THE INVENTION

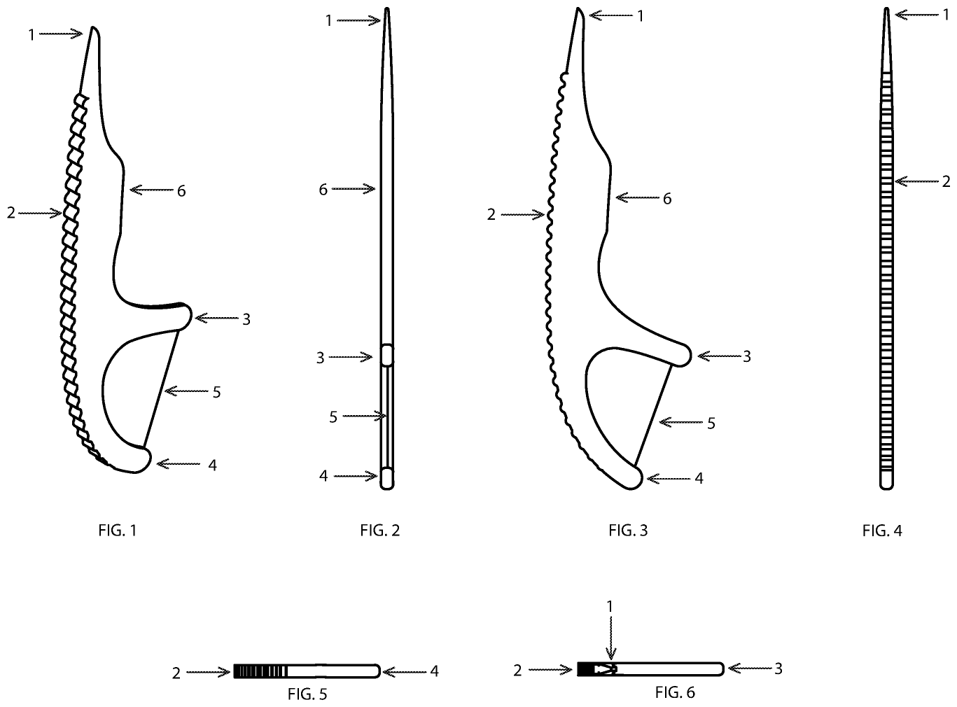

The drawings present the preferred embodiment of the invention. Variations in each of the components is possible and mentioned in some of the additional embodiments listed in the SUMMARY OF THE INVENTION SECTION. FIG. 1 shows the preferred embodiment which comprises and handle 6, a long sculpted edge on the tongue scraper 2, two forked floss tines, one toward the center of the handle 3 and one on the edge of the device 4, prongs 3 and 4 holding dental floss 5 between them and a dental floss pick 1, on the opposite end of the device from the dental floss prong 4 on the edge of the device.

I claim:

1. a parallel to face tongue scraper with a dental flosser and dental floss pick comprising:
a handle;
a radius sculpted top edge of the handle, the sculpted potion covering the majority of the top edge of the handle, with the sculpted features of the handle continuous;
a tapered dental floss pick on one side of the handle;
a dental flosser with two tines, the dental flosser in the same plane as the handle, the dental flosser being on the end of the handle opposite the dental floss pick.

2. the tongue scraper of claim 1, wherein the sculpted features of the top edge are not continuous.

3. The tongue scraper of claim 1 wherein the dental flosser is at a 90-degree angle in relation to the handle.

4. The tongue scraper of claim 1 wherein a length of dental floss extends between the two forks of the dental flosser.
